(19) United States
(12) Patent Application Publication
(10) Pub. No.: US 2018/0193107 A1
(43) Pub. Date: Jul. 12, 2018

(54) BALANCE ARM APPARATUS FOR SUPPORTING HEAVY TOOLS

(71) Applicants: ELECTRONICS AND TELECOMMUNICATIONS RESEARCH INSTITUTE, Daejeon (KR); Kyungpook National University Industry-Academic Cooperation Foundation, Daegu (KR)

(72) Inventors: Jung Wook SUH, Daegu (KR); Kyu Hyung KIM, Daegu (KR); Moon Kyu KIM, Daegu (KR); Jung Chul KIM, Daegu (KR); Tae Wuk BAE, Daegu (KR); Eun Chang CHOI, Daegu (KR)

(21) Appl. No.: 15/870,462

(22) Filed: Jan. 12, 2018

(30) Foreign Application Priority Data
Jan. 12, 2017 (KR) 10-2017-0005015
Nov. 17, 2017 (KR) 10-2017-0154020

(51) Int. Cl. A61B 90/50; A61B 34/30; A61B 34/00

(52) U.S. Cl. CPC A61B 90/50; A61B 2090/502; A61B 34/70; A61B 34/30

(57) ABSTRACT

According to an aspect of the present invention, there is provided a balance arm apparatus for supporting heavy tools, including a base and a joint unit with one side coupled to the base and configured to be operated in upward, downward, leftward, and rightward directions, and the other side coupled to a gimbal capable of changing directions of a tool or device. According to an embodiment of the present invention, it is possible to support a weight of a high load apparatus for movement with six-degrees-of-freedom by connecting a balance arm having three-or-more-degrees-of-freedom and capable of supporting the apparatus while changing a position of the apparatus, and a gimbal structure having three-or-more-degrees-of-freedom or more to an end of the balance arm capable of switching a direction of the apparatus.

### CROSS-REFERENCE TO RELATED APPLICATION

This application claims priority to and the benefit of Korean Patent Application No. 10-2017-0005015, filed on Jan. 12, 2017, and 10-2017-0154020, filed on Nov. 17, 2017, the disclosure of which is incorporated herein by reference in its entirety.

### FIELD OF THE INVENTION

The present invention relates to a balance arm apparatus for supporting heavy tools, and more particularly, to a balance arm apparatus for supporting heavy tools configured to assist in use of a heavy tool or mechanical device such that the heavy tool or mechanical device is repeatedly used at various angles.

### BACKGROUND

When hair transplantation is performed, hair follicles are collected using a strip method and the hair follicles are implanted using a manual hair transplanter.

In addition, in an extraction method of hair follicles, the strip method extracts a scalp of a patient's occiput in an elongated form, stitches the occiput, and then separates the extracted scalp into follicular units, while a follicular unit extraction (FUE) method is a non-incision method in which a thin punching machine is used to extract hair follicles directly from a scalp.

In addition, a method of hair transplantation may be divided into a method using a manual hair transplanter and a method using a tweezers.

Among them, a method in which a small slit is made in an implant area at which hair transplantation is required, and hair follicles (hair) are directly pushed into the slit hole using tweezers is mainly used in western countries.

Meanwhile, in the method using a manual hair transplanter which is widely used in Korea, there is no need to provide a separate slit, and hair follicles are not pressed while being planted. Therefore, it may be regarded as a more advanced method of hair transplantation.

However, a method of inserting (loading) a hair follicle into a needle of a conventional manual hair transplanter requires a slit in a side of the needle in order to load the hair follicle into the manual hair transplanter. In addition, since a treatment using the hair transplanter (hair transplanting machine) is performed by loading collected and separated hair follicles (hair) one by one on the needle of the hair transplanter and implanting the hair follicles one by one, at the time of treatment, multiple hair transplanters are required and it is necessary to change a hair transplanter being used each time in order to proceed with a hair transplantation task. Therefore, a treatment using the hair transplanter has a limitation on speed, which makes a degree of fatigue felt by a doctor performing the treatment and assisting nurses/auxiliaries very large. In addition, a long treatment time is also a burden on a patient undergoing the treatment.

In order to overcome the above problem, the present applicant has developed an automated hair transplanter that may sequentially change a plurality of needles loaded with hair follicles. This device has advantages in various aspects such as an operation time and a fatigue felt by an operator.

However, the automated hair transplanter (hair transplantation apparatus) is bulky and heavy when compared to the conventional manual apparatus, and thus a practitioner has problems in using the automated hair transplanter for a long time.

As a supplementary explanation, when hair transplantation is generally performed on a currently bald patient, a doctor repeats the same hair transplantation operation about 2,000 times. In this case, performing the repetitive operation while holding a heavy device weighing 1 kg or more for a long time is problematic without any additional equipment.

Furthermore, in addition to an automatic hair transplantation apparatus, there are many heavy apparatuses such as a welding machine and the like which require a user to perform a task for a long time by hand, and the apparatuses demand a load-bearing balance arm capable of supporting the weight thereof.

### SUMMARY

The present invention is directed to a balance arm apparatus for supporting heavy tools capable of being repeatedly used at various angles while supporting a heavy tool and a mechanical device such as a hair transplantation device, a welding machine, or the like.

In addition, a balance arm apparatus for supporting heavy tools capable of easily changing a direction of a tools and mechanical device of which a center of gravity is supported using a mechanical gimbal, and easily changing a position of the tool or mechanical device using a gravity compensation mechanism composed of a spring, a non-circular pulley, and the like.

According to an aspect of the present invention, there is provided a balance arm apparatus for supporting heavy tools, the balance arm apparatus including, a base and a joint unit with one side coupled to the base and configured to operate in upward, downward, leftward, and rightward directions, and the other side coupled to a gimbal which allows a direction of a tool or device to be changed.

The joint unit may include a base coupling portion rotatably coupled to the base, a joint connecting portion mounted to be spaced apart from the base coupling portion, and a working joint mounted between the base coupling portion and the joint connecting portion.

An intermediate coupling portion may further be included between the base coupling portion and the joint connecting portion, a first working joint may be mounted between the base coupling portion and the intermediate coupling portion, and a second working joint may be mounted between the intermediate coupling portion and joint connecting portion.

Tension portions that are each composed of a spring and a cable may be mounted between the base coupling portion and the intermediate coupling portion and between the intermediate coupling portion and the joint connecting portion, and a non-circular pulley may be mounted on the base coupling portion such that the base coupling portion obtains torque through a tension of the tension portion.

In the non-circular pulley, a reference point may be formed on a rotation axis of one of links constituting first and second working joints and a shape of the non-circular pulley may be formed such that an angle (θ) is in a range of 0 and 90° and, as the angle (θ) increases, a radius (R) gradually decreases.

The intermediate coupling portion may include an idler.

An extension portion may be formed in a direction perpendicular to an outer link constituting the first working joint, or an auxiliary link may be mounted on the outer link.

Weights may be mounted on the intermediate coupling portion and the joint connecting portion.

The working joint may be composed of a single link or a plurality of links, and when the working joint is composed of the plurality of links, the working joint may be formed of two parallelogrammic joints.

The gimbal may include a first spherical link coupled to a joint connecting portion constituting the joint unit, a second spherical link coupled to an end portion of the first spherical link, a first rotating joint mounted on a joint portion of the joint connecting portion and one side of the first spherical link, a second rotating joint mounted on a joint portion of the first spherical link and the second spherical link, and a third rotating joint mounted on an end portion of the second spherical link.

An angle between an axis of the first spherical link and an axis of the second spherical link may be formed in a range of 50 to 70°.

A bearing for mechanical rotation of the spherical links and a slip ring for wiring rotation of electrical wiring may be mounted in the spherical links.

The balance arm apparatus may be mounted on a movable base cart or a stationary structure.

### BRIEF DESCRIPTION OF THE DRAWINGS

The above and other objects, features, and advantages of the present invention will become more apparent to those of ordinary skill in the art by describing exemplary embodiments thereof in detail with reference to the accompanying drawings, in which:

FIG. 1 is a perspective view showing a state in which a balance arm apparatus for supporting heavy tools according to the present invention is installed;

FIGS. 2 to 5 are views showing embodiments of the balance arm apparatus for supporting heavy tools according to the present invention;

FIG. 6 is a front view showing a non-circular pulley included in the balance arm apparatus for supporting heavy tools according to the present invention;

FIG. 7 is a schematic view showing a configuration of a gimbal of the balance arm apparatus for supporting heavy tools according to the present invention;

FIG. 8 is a cross-sectional view showing an internal structure of the gimbal of the balance arm apparatus for supporting heavy tools according to the present invention;

FIGS. 9A to 9H are views showing various configuration examples of the balance arm apparatus for supporting heavy tools according to the present invention;

FIG. 10 is a flowchart showing a balance arm calibration process of the balance arm apparatus for supporting heavy tools according to the present invention which has six-degrees-of-freedom;

FIGS. 11A to 11E are view showing a balance arm calibration order of the balance arm apparatus for supporting heavy tools according to the present invention which has six-degrees-of-freedom;

FIG. 12 is a view showing an adjusting screw for calibration of the gimbal in the balance arm apparatus for supporting heavy tools according to the invention; and

FIG. 13 is an exemplary view showing a working range required in an operation using the balance arm apparatus for supporting heavy tools according to the present invention.

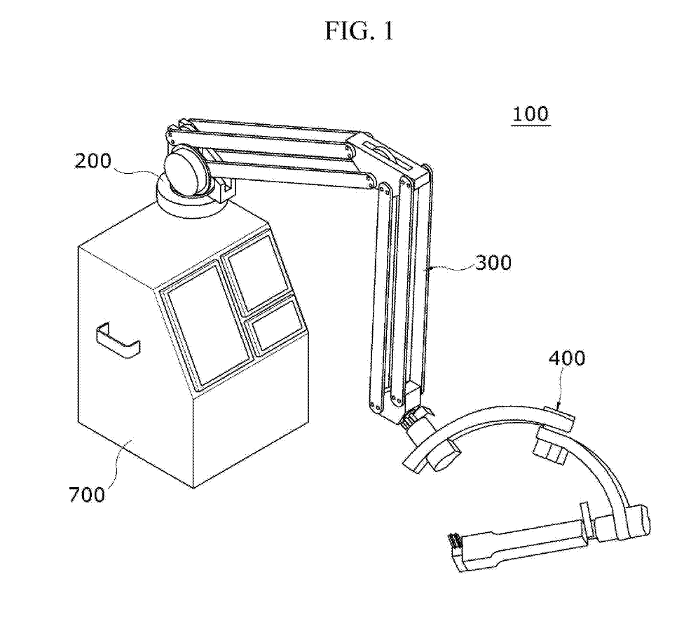

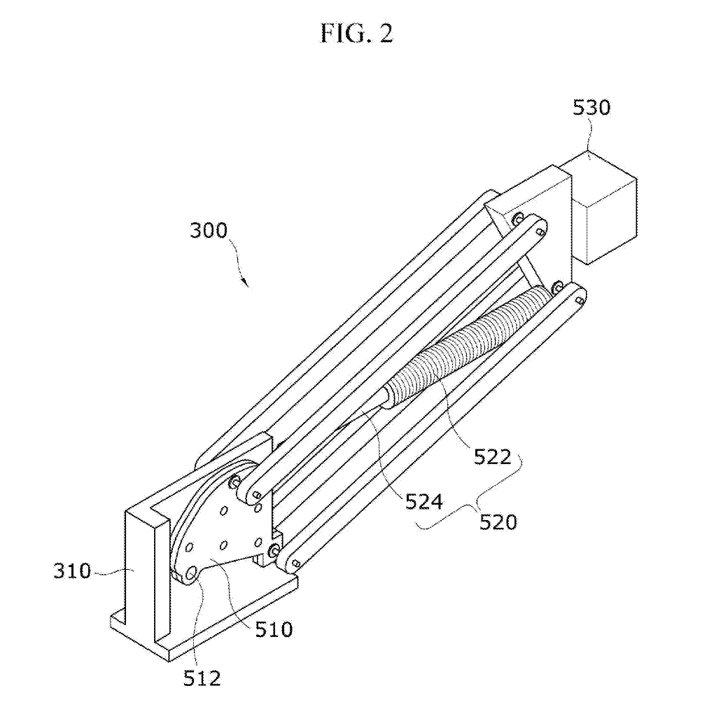

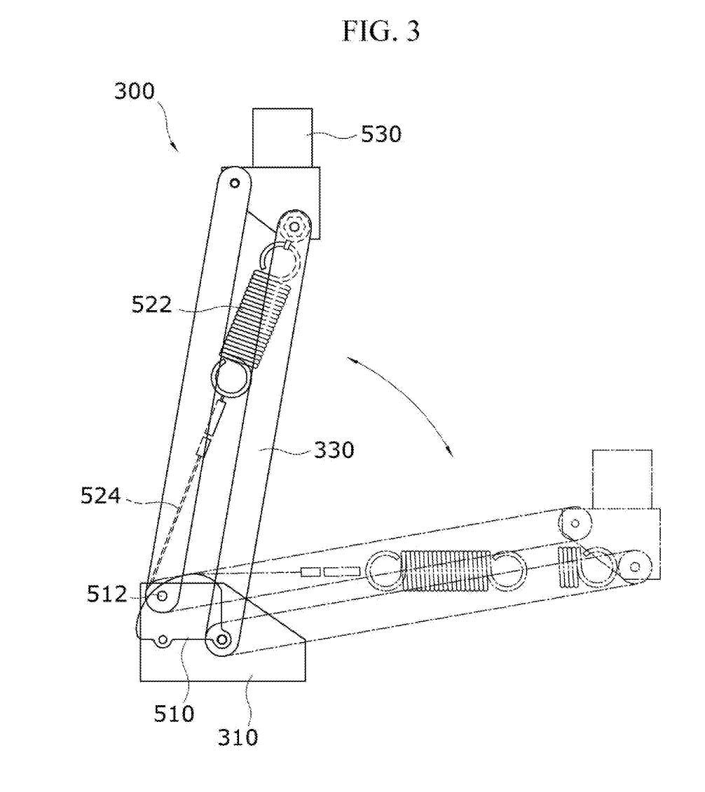

### DETAILED DESCRIPTION

Above-described advantages and features of the present invention, and methods of achieving the same will be clearly understood with reference to the accompanying drawings and the following detailed embodiments.

However the present invention is not limited to the embodiments to be disclosed, but may be implemented in various different forms. The embodiments are provided in order to fully explain the present invention and fully explain the scope of the present invention for those skilled in the art. The scope of the present invention is defined by the appended claims.

Meanwhile, the terms used herein are provided to only describe embodiments of the present invention and not for purposes of limitation. Unless the context clearly indicates otherwise, the singular forms include the plural forms. It will be understood that the terms “comprise” and/or “comprising” when used herein, specify some stated components, steps, operations and/or elements, but do not preclude the presence or addition of one or more other components, steps, operations and/or elements.

Hereinafter, the configuration of the present invention will be described with reference to the accompanying drawings.

A balance arm apparatus 100 for supporting heavy tools (hereinafter, referred to as a “balance arm apparatus”) of the present invention includes a base 200, a joint unit 300 with one side coupled to the base 200, and a gimbal 400 to which the other side of the joint unit 300 is coupled.

Here, the balance arm apparatus 100 is mounted on a base cart 700 with wheels for smooth movement and storage as well as mounted on a fixed structure such as a ceiling or a table. In the present invention, an example of the balance arm apparatus 100 mounted on the base cart 700 will be described.

In this case, the base cart 700 includes a cart case in which components such as a personal computer (PC), a power source, a control box, and the like are mounted, moving wheels disposed to be spaced a distance from each other under the cart case and having stoppers, and a controller mounted on an outer side of the cart case.

The base 200 has a predetermined thickness and is mounted on the base cart 700.

Here, the base 200 is formed in various shapes according to an environment, a purpose, or the like. In the present invention, the base 200 is formed in a plate shape having a predetermined thickness and size.

Further inside the base 200, a bearing, a roller, a rotation shaft, and a driving motor, which are known for rotational operation, are configured alone or in combination, and a description thereof will be omitted.

The one side of the joint unit 300 is coupled to the base 200, and the other side of the joint unit 300 is selectively coupled to the gimbal 400 capable of changing tools or devices or a direction of the tools or devices.

Here, an example of the gimbal 400 capable of changing tools or devices or a direction of the tools or devices mounted at the other side of the joint unit 300 will be described.

That is, the one side of the joint unit 300 is mounted on the base 200, and the other side of the joint unit is coupled to the gimbal 400 so that the gimbal 400 is operated with three-degrees-of-freedom, that is, in X, Y, and Z directions.

Further, the joint unit 300 includes a base coupling portion 310 coupled to the base 200, a joint connecting portion 320 provided to be spaced apart from the base coupling portion 310, and a working joint 330 mounted between the base coupling portion 310 and the joint connecting portion 320.

That is, in the joint unit 300, the base coupling portion 310 is disposed to be spaced a distance from the joint connecting portion 320, the working joint 330 is mounted between the base coupling portion 310 and the joint connecting portion 320, and thus the joint unit 300 may be operated with three-degrees-of-freedom.

The joint unit 300 further includes an intermediate coupling portion 340 disposed between the base coupling portion 310 and the joint connecting portion 320. A first working joint 332 is mounted between the base coupling portion 310 and the intermediate coupling portion 340, and a second working joint 334 is mounted between the intermediate coupling portion 340 and the joint connecting portion 320.

That is, the intermediate coupling portion 340 is disposed between the base coupling portion 310 and the joint connecting portion 320, the first working joint 332 is mounted between the base coupling portion 310 and the intermediate coupling portion 340, and a second working joint 334 is mounted between the intermediate coupling portion 340 and the joint connecting portion 320.

Tension portions 520 composed of a spring 522 and a cable 524 are mounted between the base coupling portion 310 and the intermediate coupling portion 340 and between the intermediate coupling portion 340 and the joint connecting portion 320, and a non-circular pulley 510 is mounted on the base coupling portion 310 so that the base coupling portion 310 may obtain a torque through a tension of the tension portion 520.

That is, a desired amount of torque may be obtained by a tension of the spring 522 and the cable 524 while the working joint 330 is angularly deformed by the non-circular pulley 510.

Here, one or more coupling pins 512 or fixing pins are mounted on the non-circular pulley 510 to prevent rotation thereof due to torque generated by a tension of the cable 524, and a correct position of the non-circular pulley 510 may also be maintained.

In this case, one end of the tension portion 520 is wound around the base coupling portion 310 and fixedly coupled to the intermediate coupling portion 340, while the other end of the tension portion 520 is fixedly coupled to the joint connecting portion 320 to be rotated with respect to one point of the intermediate coupling portion 340.

In addition, a turnbuckle may be selectively mounted on the tension portion 520 to adjust the tension thereof, and a spring fixing link or the like may be included in the tension portion 520 for fixing the tension portion 520 as necessary.

In the non-circular pulley 510, a reference point is formed on a rotation axis of one of the links constituting the working joint 330 and a shape thereof is formed such that an angle θ is in a range of 0 and 90° and, as the angle θ increases, a radius R gradually decreases, but the relationship therebetween is not linear.

For example, when the joint connecting portion 320 and the working joint 330 constituting the joint unit 300 are raised relative to the non-circular pulley 510 of the base coupling portion 310, the amount of torque (tension×moment arm length) applied to the joint unit 300 is reduced, and when the joint connecting portion 320 and the working joint 330 are lowered relative to the non-circular pulley 510 of the base coupling portion 310, the amount of torque is increased as the joint unit 300 moves downward (to the right) and approaches the horizontal.

When a horizontal posture refers to 0°, in the cause in which torque required for balancing the joint unit 300 is proportional to a cosine (cos) value and the joint unit 300 is further lowered to have an angle of 0 to −90°, a tension applied to the cable 524 by the spring 522 is increased while a moment arm (the shortest distance from the center of rotation to the cable) is reduced and the torque applied to the joint unit 300 is reduced. Accordingly, the torque is proportional to the cosine value.

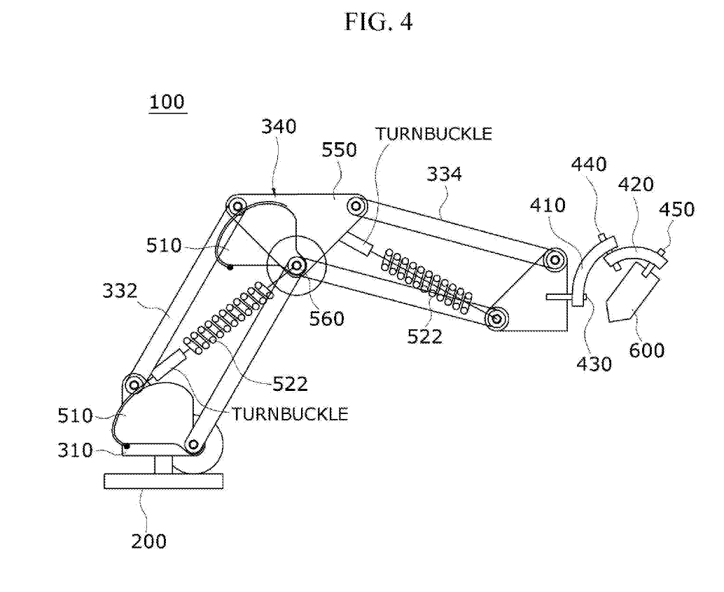

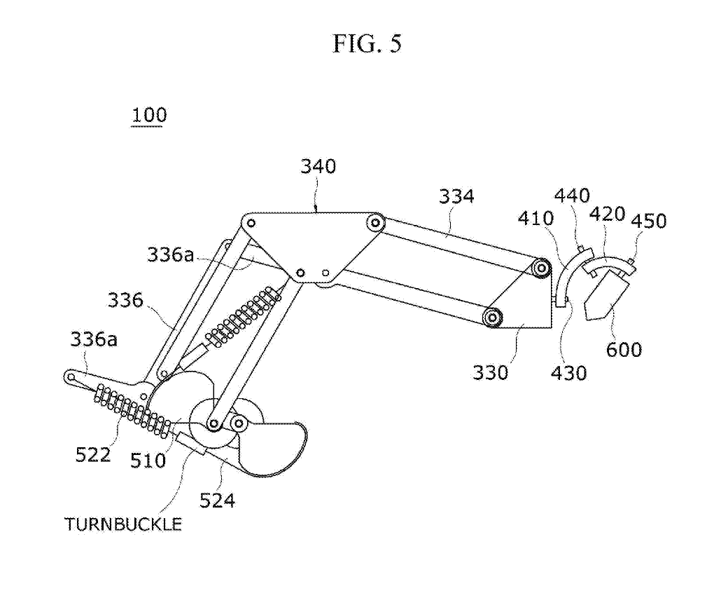

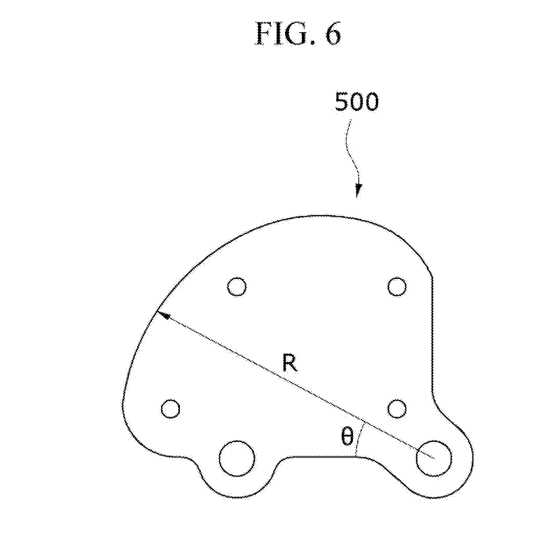

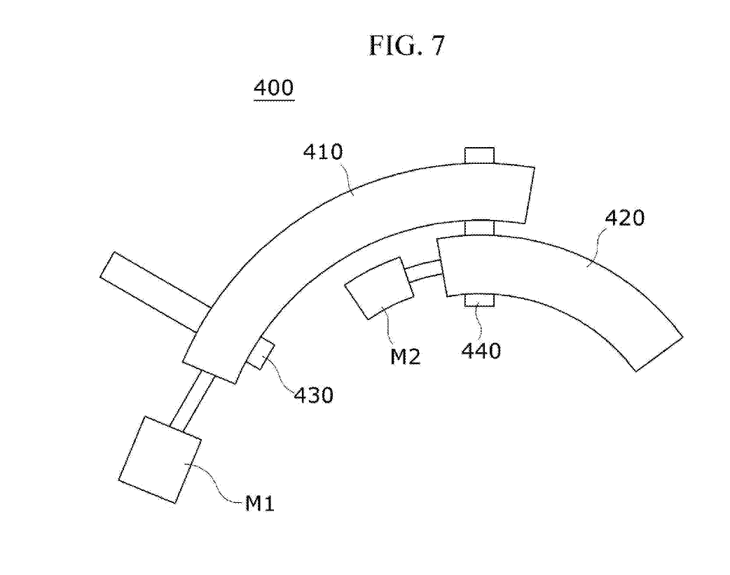

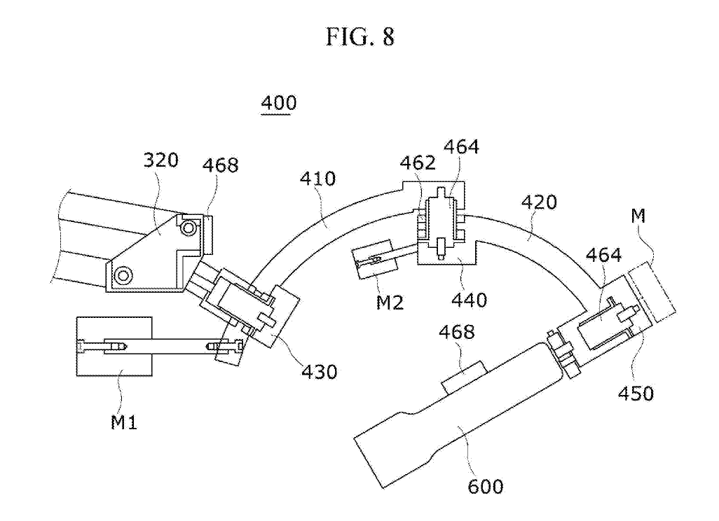

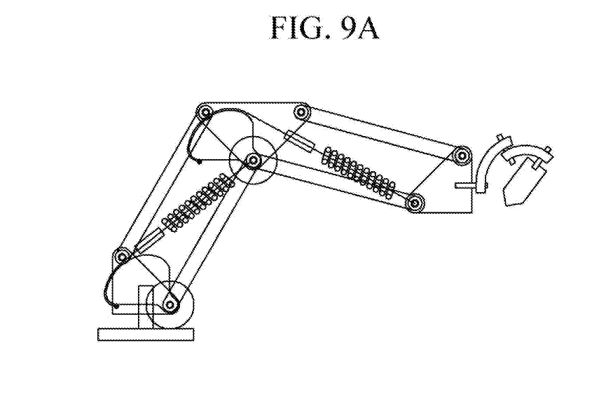

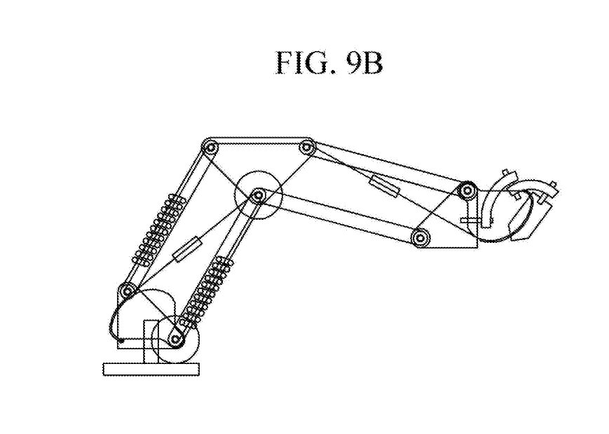

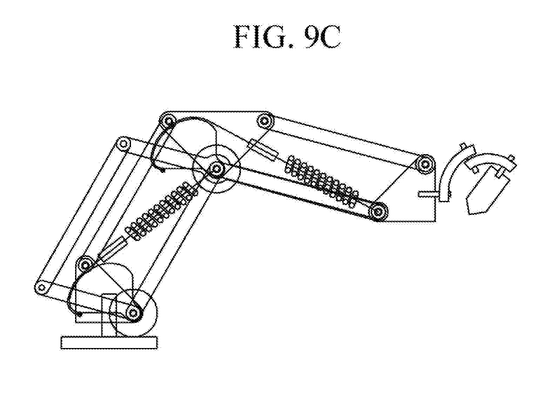

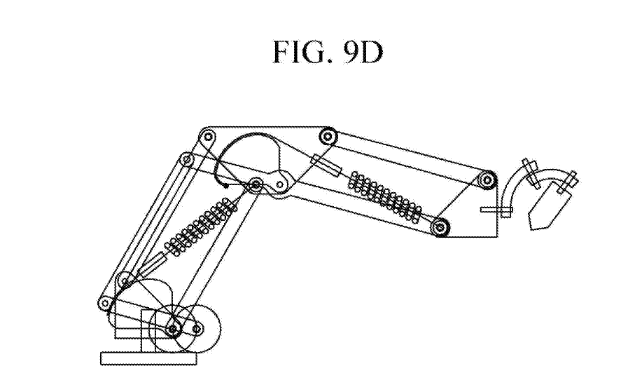

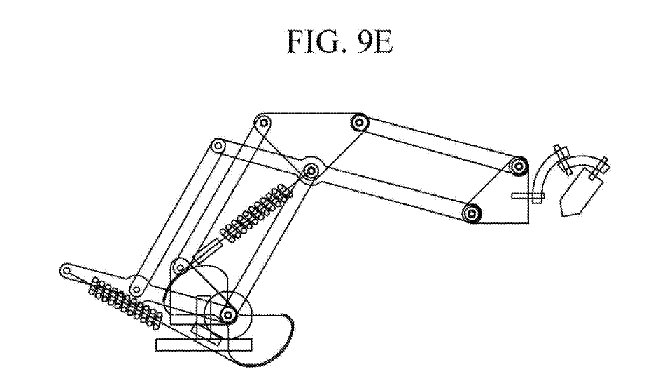

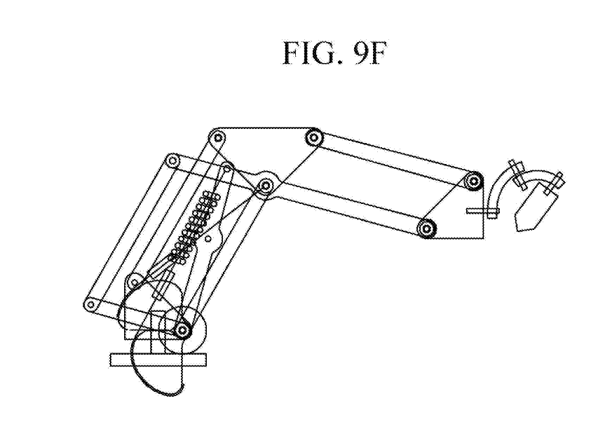

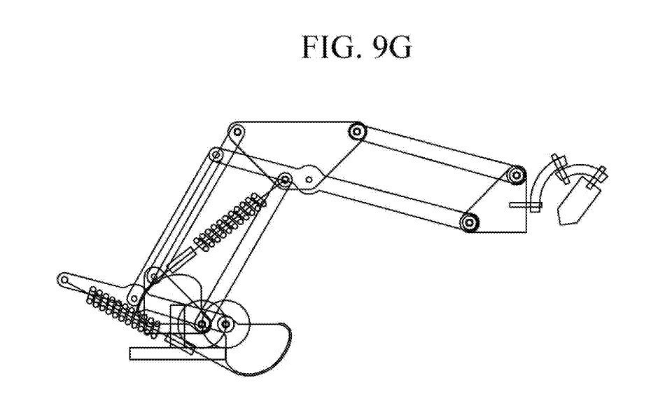

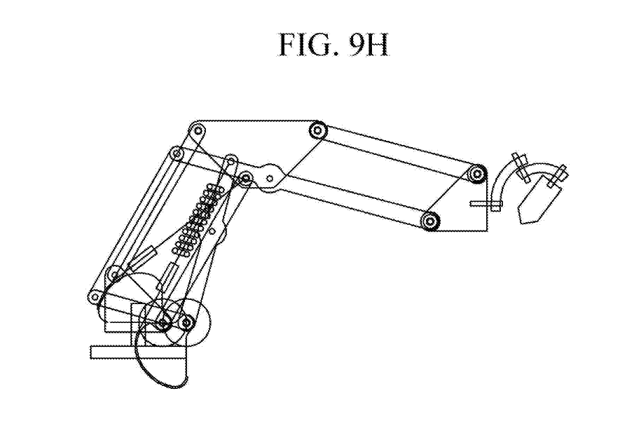

Next, the joint unit 300 may be configured as shown in FIGS. 4 and 5.

First, the joint unit 300 shown in FIG. 4 may change a path of the cable 524 and the spring 522, and a length of the working joint 330 and a weight of the spring 522 may be reduced by an idler 550 being mounted on the intermediate coupling portion 340.

The joint unit 300 shown in FIG. 5 further includes an extension portion 336a formed in a direction perpendicular to an outer link 336 constituting the first working joint 332, or the auxiliary link is mounted in a direction perpendicular to the joint unit 300.

Here, the non-circular pulley 510, the tension portion 520, the spring, a brake or the like, which constitute a gravity compensation mechanism, may be disposed on a base of the outer link 336 so that a load required for gravity compensation may be reduced and components such as the spring and the like may be lightened, resulting in a reduction of an overall inertia, thereby reducing an overall inertia felt by the user.

In addition, a weight 530 configured to calibrate (adjust) the gravity compensation mechanism may be mounted on the joint connecting portion 320. Here, adjustment of the weight 530 is performed together with adjustment of an initial tension of the tension portion 520.

Also, a brake 560 or a servo-motor may be mounted on the intermediate coupling portion 340 and the joint connecting portion 320 so that the brake 560 or the servo-motor may be fixed at a desired angle.

Here, when the servo-motor is applied to the intermediate coupling portion 340 and the joint connecting portion 320, a robot arm (maniplator) having a balancing function is implemented.

The working joints 330, 332, and 334 may be composed of a single link or a plurality of links, and here, in the present invention, the working joints 330, 332, and 334 include two parallelogrammic joints.

The gimbal 400 is mounted on the other side of the joint unit 300, and moves in the forward, backward, leftward, and rightward directions.

That is, the gimbal 400 is coupled to the joint connecting portion 320 constituting the joint unit 300 to provide fine movement to a tool or mechanical device thereon.

In addition, the gimbal 400 is moved to a position set primarily through the joint unit 300 and is then operated while moving in pitch, yaw, and roll directions in addition to the X, Y, Z directions.

The gimbal 400 includes a first spherical link 410 coupled to the joint connecting portion 320, a second spherical link 420 coupled to an end portion of the first spherical link 410, a first rotating joint 430 mounted on the joint connecting portion 320 and a joint portion of one side of first spherical link 410, a second rotating joint 440 mounted on a joint portion of the first spherical link 410 and the second spherical link 420, and a third rotating joint 450 mounted on an end portion of the second spherical link 420.

That is, in the first spherical link 410, one end portion of the first spherical link 410 is coupled to the joint connecting portion 320 via the first rotating joint 430, and one end portion of the second spherical link 420 is disposed at and coupled to the other end portion of the first spherical link 410 via the second rotating joint 440, the third rotating joint 450 is coupled to the other end portion of the second spherical link 420.

Here, an angle between an axis of the first spherical link 410 and an axis of the second spherical link 420 is formed in a range of 50 to 70° so that the device may be easily accessed at any angle.

The joint portion of the gimbal 400 is subjected to a weight or a calibration process for gravity compensation or counter-balancing.

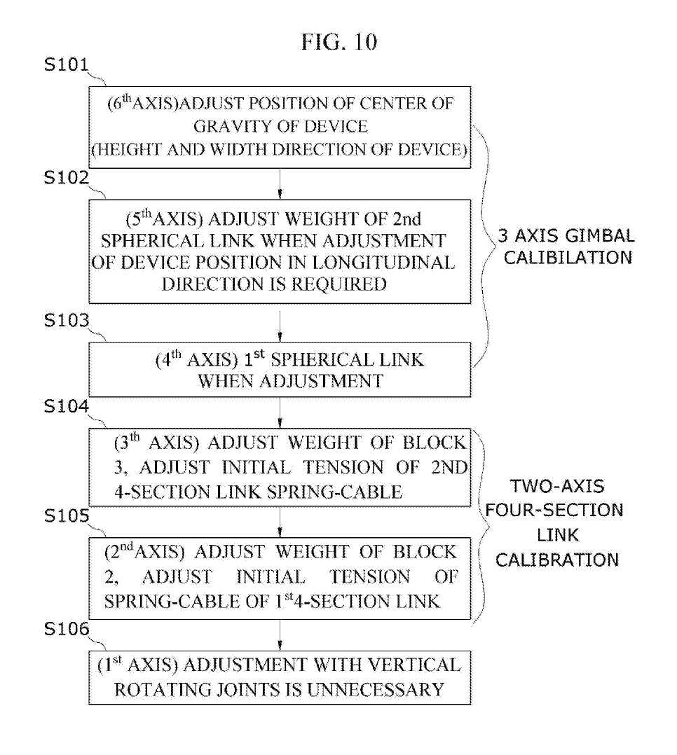

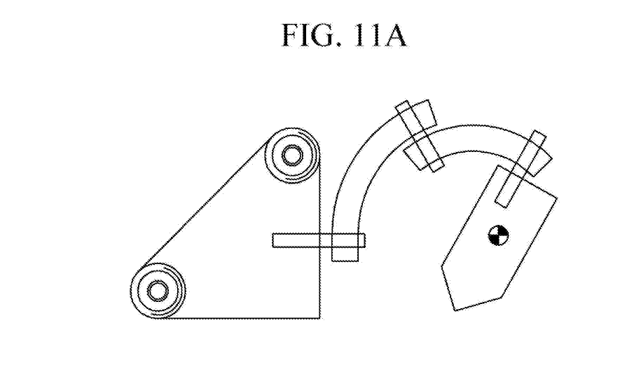

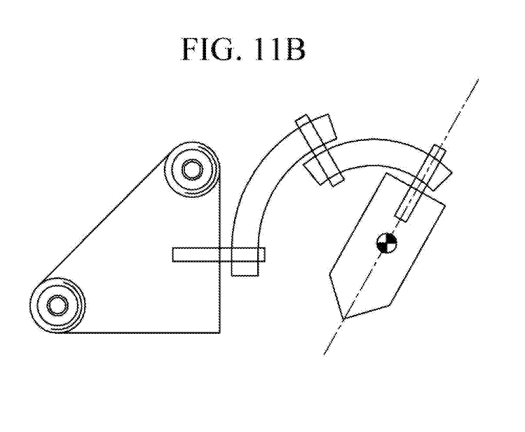

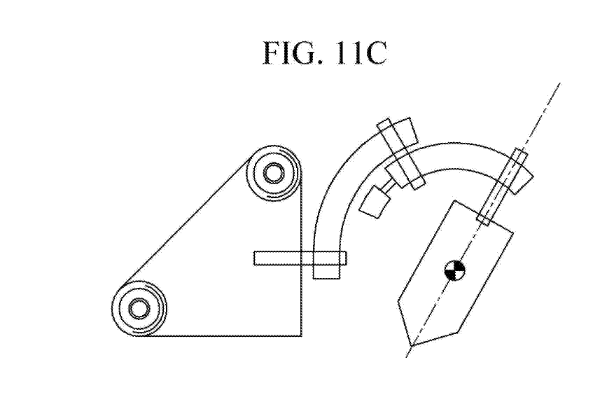

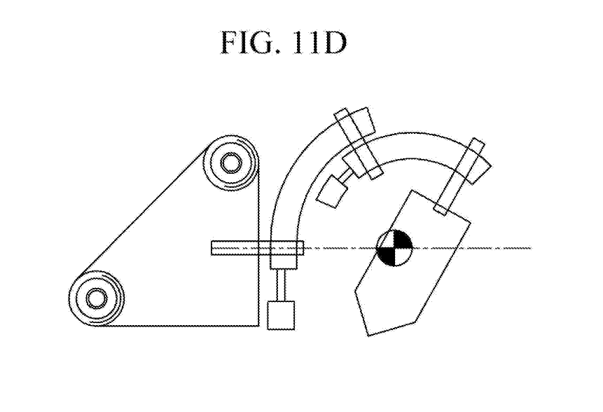

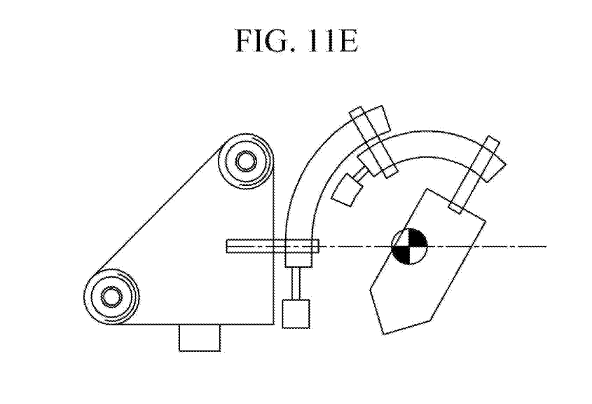

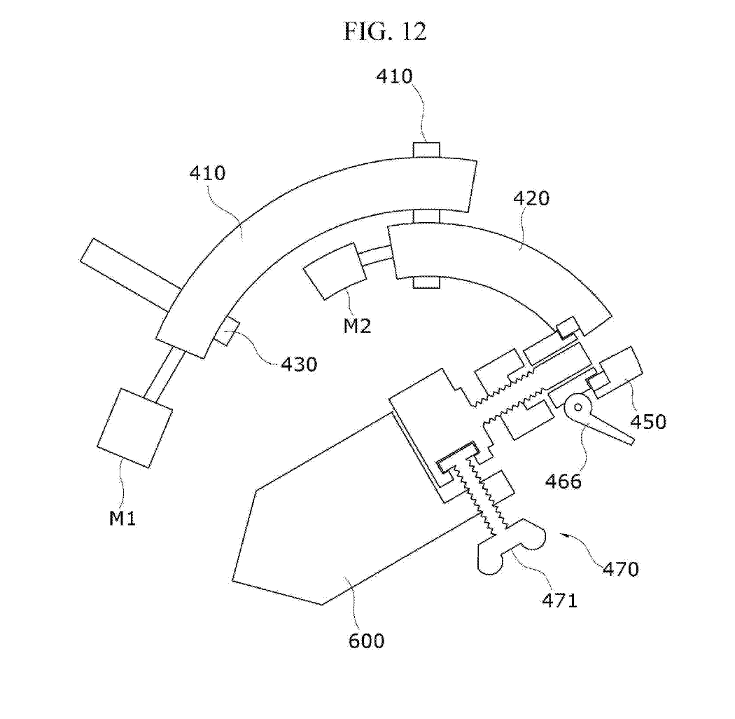

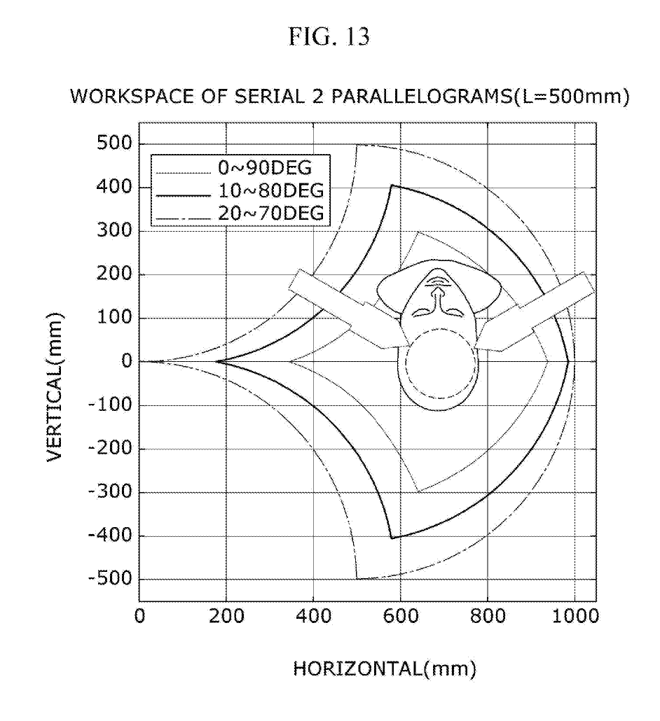

Referring to FIG. 10, which is a flowchart showing a balance arm calibration process of the balance arm apparatus for supporting heavy tools according to the present invention which has six-degrees-of-freedom, and FIGS. 11A to 11E are view showing a balance arm calibration order of the balance arm apparatus for supporting heavy tools according to the present invention which has six-degrees-of-freedom, focusing on a gimbal portion. Here, the calibration is preferably performed from a distal joint of the balance arm apparatus.

First, In, S101, when a center of gravity of a distal device 600 is located on a rotation axis of the third rotating joint 450 mounted on the end of the second spherical link 420, the distal device 600 may be freely rotated relative to the third rotating joint 450.

Here, the fact that the distal device 600 may be rotated freely means that a posture thereof is maintained at any desired posture or continuously rotated in a direction rotated by inertia even when there is no friction in each joint.

In this case, the distal device 600 may be additionally equipped with a 2-degree-of-freedom (X and Y) controller or a weight to control a position of an axis located on a rear side of the distal device 600.

Next, in steps S102 and S103, when centers of gravity of the second spherical link 420 and the distal device 600, which are the parts rotated by the second rotary rotating 440, are located on a rotation axis of the second rotating joint 440, the distal device 600 and the second spherical link 420 are allowed to operate while being rotated freely relative to the second and third rotating joints 440 and 450.

In this case, a weight M2 is selectively mounted on the distal device 600 and the second spherical link 420 configured to adjust a position thereof in a longitudinal direction, and in the third rotating joint 450, a weight M (not shown) above the distal device 600 may be adjusted in the same manner as the calibration.

In, S104, Likewise, a weight and position of a weight M1 on the first spherical link 410 may be adjusted by calibrating a rotation axis of the first rotating joint 430.

Accordingly, there should be portions in the two spherical links 410 and 420 that may adjust the weight and position of each of the weights M and the rotation axis of the third rotating joint 450, and the rotation axis of the third rotating joint 450.

Next, in the calibration process for the second working joint 334, adjustment of a separate weight (mark as a square in the lower part of the joint connecting portion 2 in FIG. 11E) and adjustment of the (initial) tension of the tension portion 520 are simultaneously performed.

In, S105, the weight M (not shown separately) on the intermediate coupling portion may be adjusted, and the (initial) tension of the tension portion 520 may be adjusted by calibrating the first working joint 332.

To this end, there is a portion of the intermediate coupling portion 340 and the joint connecting portion 320 that may adjust a weight, and the tension of the tension portion 520 is adjusted via a tension adjusting device such as a turnbuckle.

In, S106, Here, since the base coupling portion 310 is a joint that rotates with respect to a vertical direction, gravity compensation is unnecessary.

Next, a bearing 462 for mechanical rotation of the first spherical link 410 and the second spherical link 420, and a slip ring 464 for wiring rotation of electrical wiring are disposed on the first spherical link 410 and the second spherical link 420.

As an example, the gimbal 400 may be separated from the arm part and the distal device part for disinfection and the like, and a quick-release (QR) clamp 466 for simple engagement with a fixing shaft and a connector 468 for wiring connection are applied to both sides of the gimbal 400 for such separation.

In addition, the weight M for calibration is composed of a single thin film or a plurality of thin films for fine adjustment.

The gimbal 400 includes an adjustment screw 470 for calibration based on the second rotating joint 440 and the third rotating joint 450.

First, the center of gravity of the distal device 600 is aligned with the rotation axis of the third rotating joint 450 using a rotation axis position adjusting bolt 471, and then a position of the center of gravity of the distal device 600 including the second spherical link 420 and the like is aligned with the rotation axis of the second rotating joint 440 using a device longitudinal position adjusting nut 472.

Here, when necessary, a first weight adjustment M1 may be used, and then a second weight adjustment M2 may be adjusted so that the center of gravity of the distal device 600 including the gimbal 400 is aligned with the rotation axis of the first rotating joint 430.

As a result, the center of gravity of the distal device 600 together with the components such as the first and second spherical links 410 and 420 are always aligned with the three rotation axes so that a required posture may be maintained even when there is no friction in each joint.

As described above, according to the embodiment of the present invention, it is possible to support a weight of a high load apparatus for movement with six-degrees-of-freedom by connecting a balance arm having three-or-more-degrees-of-freedom and capable of supporting the apparatus while changing a position of the apparatus, and a gimbal structure having three-or-more-degrees-of-freedom to an end of the balance arm capable of switching a direction of the apparatus.

Further, a mechanism such as a parallelogrammic link, to which gravity compensation is easily applied, is applied to a large articulated structure of a balance arm for positioning, and thus it is possible to prevent the apparatus from falling down due to the weight of the apparatus or rotating the apparatus.

While the present invention has been particularly described with reference to exemplary embodiments, it will be understood by those of skilled in the art that various changes in form and details may be made without departing from the spirit and scope of the present invention.

Therefore, the exemplary embodiments should be considered in a descriptive sense only and not for purposes of limitation.

The scope of the invention is defined not by the detailed description of the invention but by the appended claims, and encompasses all modifications and equivalents that fall within the scope of the appended claims.

What is claimed is:

1. A balance arm apparatus for supporting heavy tools, comprising:
a base; and
a joint unit with one side coupled to the base and configured to operate in upward, downward, leftward, and rightward directions, and the other side coupled to a gimbal which allows a direction of a tool or device to be changed.

2. The balance arm apparatus of claim 1, wherein the joint unit comprises:
a base coupling portion rotatably coupled to the base;
a joint connecting portion mounted to be spaced apart from the base coupling portion; and
a working joint mounted between the base coupling portion and the joint connecting portion.

3. The balance arm apparatus of claim 2, wherein:
an intermediate coupling portion is further included between the base coupling portion and the joint connecting portion;
a first working joint is mounted between the base coupling portion and the intermediate coupling portion; and
a second working joint is mounted between the intermediate coupling portion and joint connecting portion.

4. The balance arm apparatus of claim 3, wherein:
tension portions that are each composed of a spring and a cable are mounted between the base coupling portion and the intermediate coupling portion and between the intermediate coupling portion and the joint connecting portion; and
a non-circular pulley is mounted on the base coupling portion such that the base coupling portion obtains torque through a tension of the tension portion.

5. The balance arm apparatus of claim 4, wherein, in the non-circular pulley, a reference point is formed on a rotation axis of one of links constituting first and second working joints and a shape of the non-circular pulley is formed such that an angle (θ) is in a range of 0 and 90° and, as the angle (θ) increases, a radius (R) gradually decreases.

6. The balance arm apparatus of claim 3, wherein the intermediate coupling portion includes an idler.

7. The balance arm apparatus of claim 3, wherein an extension portion is formed in a direction perpendicular to an outer link constituting the first working joint, or an auxiliary link is mounted on the outer link.

8. The balance arm apparatus of claim 3, wherein weights are mounted on the intermediate coupling portion and the joint connecting portion.

9. The balance arm apparatus of claim 2, wherein the working joint is composed of a single link or a plurality of links, and when the working joint is composed of the plurality of links, the working joint is formed of two parallelogrammic joints.

10. The balance arm apparatus of claim 1, wherein the gimbal comprises:
a first spherical link coupled to a joint connecting portion constituting the joint unit;
a second spherical link coupled to an end portion of the first spherical link;
a first rotating joint mounted on a joint portion of the joint connecting portion and one side of the first spherical link;
a second rotating joint mounted on a joint portion of the first spherical link and the second spherical link; and
a third rotating joint mounted on an end portion of the second spherical link.

11. The balance arm apparatus of claim 10, wherein an angle between an axis of the first spherical link and an axis of the second spherical link is formed in a range of 50 to 70°.

12. The balance arm apparatus of claim 11, wherein a bearing for mechanical rotation of the spherical links and a slip ring for wiring rotation of an electrical wiring are mounted in the spherical links.

13. The balance arm apparatus of claim 1, wherein the balance arm apparatus is mounted on a movable base cart or a stationary structure.
